(19) United States
(12) Patent Application Publication
(10) Pub. No.: US 2009/0018423 A1
(43) Pub. Date: Jan. 15, 2009

(54) PATCH FOR EXTRACTING GLUCOSE

(75) Inventors: Kwang-Jae Choi, Seoul (KR); Tae-Ho Kim, Gyeonggi-do (KR); Seung-Ro Lee, Daegu (KR)

Correspondence Address: MEREDITH & KEYHANI, PLLC, 330 MADISON AVE., 6TH FLOOR, NEW YORK, NY, 10017, US

(73) Assignee: KMH CO., LTD of 405

(21) Appl. No.: 11/995,867

(22) PCT Filed: Jul. 14, 2006
(86) PCT No.: PCT/KR06/02770

§ 371 Date: Jul. 30, 2008

(30) Foreign Application Priority Data
Jul. 19, 2005 (KR) 10-2005-0065302

(51) Int. Cl. A61B 5/145

(52) U.S. Cl. 600/347

(57) ABSTRACT

Disclosed herein is a patch for extracting glucose. The patch of the present invention includes a frame (310), a first electrode film (320), two media (330a, 330b), a second electrode film (350), and a support (360). The frame (310) includes two protrusions formed thereon, the two protrusions having contact holes. The first electrode film (320) is attached to an inner side of the frame, and is provided with holes corresponding to the contact holes, the first electrode film including electrodes, a terminal, and copper wires. The two media (330a, 330b) are accommodated in the protrusions and have ionic conductivity and hydrophilic properties, at least one of the two media including enzymes. The second electrode film (350) includes electrodes for extracting glucose, a terminal for realizing electrical connection and copper wires for connecting the electrodes to the terminal. The support (360) is attached to the second electrode film.

### TECHNICAL FIELD

The present invention relates, in general, to a patch for extracting glucose, which is used for a glucose measurement device for measuring glucose.

### BACKGROUND ART

Generally, diabetes is an incurable disease occurring when glucose, produced through the digestion of food, is accumulated in blood in a human body due to a deficiency of secretion of insulin or decrease of cellular reactivity to insulin, and induces complications, including cardiovascular diseases, such as hardening of the arteries, high blood pressure, or cerebrovascular infarction, kidney diseases, such as diabetic nephropathy, eye diseases, such as diabetic retinitis or cataract, skin diseases, such as pyoderma or gangrene, or oral diseases, such as paradentitis. With the realization of social and economic development, a diabetic population is currently remarkably increasing due to overeating, insufficient exercise, increased stress, etc. In the case of advanced countries, it is predicted that 5 to 10% of a total population are or may become diabetic. Currently, about 50% of diabetics do not recognize that they are diabetic. Such diabetics are recommended to basically manage their blood sugar levels by periodically monitoring their fasting blood sugar levels (maximum tolerance of 140 mg/dL) and their two hours post-meal blood sugar levels (maximum tolerance of 200 mg/dL). In addition to the above items, variation in blood sugar may be severe over the course of a day due to a plurality of conditions, such as general physical condition, the type and amount of food taken, age, existence of complications, stress, or other accompanying diseases. Accordingly, self-examination and self-management of blood sugar levels by diabetics can be considered very important factors for maintaining diabetics' health and preventing complications.

In the prior art, a method of monitoring glucose in the blood, that is, blood sugar, has depended on a blood gathering method. That is, a blood gathering method is a method of gathering blood using a blood lancet on the tip of a finger, or a specific region of a body, and is disadvantageous in that only a one-shot inspection is enabled. Further, a blood gathering-type blood sugar monitoring system is inconvenient in that it is impossible to measure a blood sugar level at short intervals due to pain or discomfort during blood gathering.

In consideration of such problems, various methods of measuring the concentration of components analyzed from blood without gathering blood have been developed. For example, U.S. Pat. No. 5,267,152 filed by Yang, et al. discloses non-invasive technology for measuring a blood glucose concentration using near-infrared radiation diffuse-reflection laser spectroscopy. However, such technology is not commercialized yet due to technical problems thereof.

Further, U.S. Pat. No. 5,279,543 filed by Glikfeld discloses technology for sampling a substance in a non-invasive method through a reservoir means on a skin surface using iontophoresis (electroosmosis). This technology proposes that the sampling method can be combined with a specific glucose biosensor or a glucose selective electrode so as to monitor blood glucose. However, this method is disadvantageous in that it cannot propose a structure allowing convenient use by patients, and it is difficult to continuously monitor blood sugar levels.

Further, a conventional glucose measurement apparatus including a patch for extracting glucose has been developed. For example, Korean Patent Registration No. 10-453-483 discloses a patch for a glucose extraction apparatus and method of manufacturing the patch. This scheme is described below with reference to the attached drawing.

FIG. 1 is a view showing the appearance shape of a conventional glucose measurement apparatus. As shown in FIG. 1, a display unit for displaying information such as a blood sugar level, and an input unit for inputting or selecting information are provided on the front surface of the glucose measurement apparatus.

FIG. 2 is a view showing the conventional glucose measurement apparatus with the rear part thereof detached therefrom. As shown in FIG. 2, a disposable patch 200 attached to the skin of a patient to extract glucose is provided on the rear surface of the glucose measurement apparatus. In relation to this structure, a locker 102 for locking the disposable patch, battery receiving parts 101a and 101b for receiving batteries, and terminals 103 for electrically connecting to the terminals 211 of the disposable patch 200, are provided in the glucose measurement apparatus.

FIG. 3 is an exploded perspective view showing the construction of the conventional patch 200 for extracting glucose. The patch 200 includes two hydrophilic gel discs 220a and 220b, respectively including enzymes capable of generating hydrogen peroxide through reaction with glucose, a frame 230 for accommodating the gel discs 220a and 220b, a film 240 for supporting the gel discs 220a and 220b, and a print circuit 210. On the print circuit 210, electronic electrodes used to extract glucose are printed. However, in the prior art, since electrodes are arranged on a single print circuit 210, there are problems in that connection between electrodes and terminals is complicated, conductivity is decreased due to high resistance resulting from the complicated connection, sensitivity is deteriorated, and the time required for analysis is increased.

### DISCLOSURE OF INVENTION

### Technical Problem

Accordingly, the present invention has been made keeping in mind the above problems, and an object of the present invention is to provide a patch for extracting glucose, which is provided with two electrode films, each having electrodes printed thereon, so as to reduce resistance and improve conductivity and sensitivity.

### Technical Solution

In order to accomplish the above object, the present invention provides a patch for extracting glucose, comprising a frame including two protrusions formed thereon, the two protrusions having contact holes that are formed at center portions thereof and come into contact with skin, a first electrode film attached to an inner side of the frame in a same shape as that of the frame, and provided with holes formed at locations corresponding to the contact holes, the first electrode film including an electrode for extracting glucose, a terminal for realizing electrical connection, and copper wires for connecting the electrodes to the terminal, two media accommodated in the protrusions, respectively, and adapted to have ionic conductivity for movement of ions and hydrophilic properties, at least one of the two media including enzymes required to generate ions through reaction with glucose, a second electrode film including electrodes placed at locations corresponding to the two media, respectively, to extract glucose, a terminal for realizing electrical connection, and copper wires for connecting the electrodes to the terminal, and a support attached to the second electrode film and required for physical connection to a glucose measurement device.

Preferably, the first electrode film may comprise a reference electrode used as a reference potential when current generated by reaction of the media with extracted glucose is measured, and a counter electrode used to measure current generated by reaction of the media with extracted glucose, and the electrodes of the second electrode film may comprise first and second extraction electrodes for extracting glucose from skin, and a working electrode for applying voltage that is used to measure current generated by reaction of the media with extracted glucose.

Preferably, the counter electrode may be formed in a shape of a ring, in which a hole corresponding to one of the contact holes is formed at a center portion, and may be placed at a location corresponding to one of the media, the reference electrode may be formed in a shape of a bar that is curved along an outer side of the counter electrode, the working electrode may be formed in a shape of a circle, and may be placed at a location corresponding to the counter electrode, the first extraction electrode may be formed in a shape of a partially cut ring, and may be formed to enclose an outer side of the working electrode, and the second extraction electrode may be formed in a shape of a ring, and may be placed at a location corresponding to a medium, other than the medium corresponding to the counter electrode.

Preferably, the at least one medium including enzymes may comprise a hydrophilic gel element and a nanofiber mesh sheet adhered to a bottom of the hydrophilic gel element and having enzymes attached thereto.

Preferably, the nanofiber mesh sheet may be formed in a shape of a circle having a diameter greater than that of the first extraction electrode, a center of the circle being placed at a location coincident with a center of the working electrode.

According to another embodiment of the present invention, the electrode of the first electrode film may comprise a working electrode for applying voltage that is used to measure current generated by reaction of the media with extracted glucose, and the electrodes of the second electrode film may comprise a reference electrode used as a reference potential when current generated by reaction of the media with extracted glucose is measured, a counter electrode used to measure current generated by reaction of the media with extracted glucose, and first and second extraction electrodes used to extract glucose from skin.

Preferably, the working electrode may be formed in a shape of a ring, in which a hole corresponding to one of the contact holes is formed at a center portion, and may be placed at a location corresponding to one of the media, the counter electrode may be formed in a shape of a circle having a partially recessed edge, and may be placed at a location corresponding to the working electrode, the reference electrode may be placed in a location at which the edge of the counter electrode is partially recessed, the first extraction electrode may be formed in a shape of a partially cut ring, and may be formed to enclose an outer side of the counter electrode, and the second extraction electrode may be formed in a shape of a ring, and may be placed at a location corresponding to a medium, other than the medium corresponding to the working electrode.

Preferably, the at least one medium including enzymes may comprise a hydrophilic gel element and a nanofiber mesh sheet adhered to a bottom of the hydrophilic gel element and having enzymes attached thereto.

Preferably, the nanofiber mesh sheet may be formed in a shape of a circle having a diameter greater than that of the first extraction electrode, a center of the circle being placed at a location coincident with a center of the working electrode.

Preferably, the first electrode film and the second electrode film may be constructed so that the electrodes, the terminals and the copper wires are printed on opposite surfaces thereof, and a portion of the first electrode film, other than the protrusions, is adhered to the second electrode film. In this case, preferably, the portion of the first electrode film, other than the protrusions, may be adhered to the second electrode film so that the terminal of the first electrode film is electrically connected to the terminal of the second electrode film.

Preferably, a portion of the first electrode film, in which the terminal is placed, may protrude from a remaining portion of the first electrode film, thus forming a terminal protrusion, and a portion of the second electrode film, in which the terminal is placed, may protrude from a remaining portion of the second electrode film, thus forming a terminal protrusion.

Preferably, the terminal protrusion of the second electrode film may protrude further than the terminal protrusion of the first electrode film. In this case, preferably, the portion of the second electrode film, in which the terminal is placed, may be bent at an edge portion of the support and be adhered to a bottom surface of the support, thus causing the terminal to be exposed from the back surface of the support.

### BRIEF DESCRIPTION OF THE DRAWINGS

FIG. 1 is a view showing the appearance of a conventional glucose measurement apparatus;

FIG. 2 is a view showing the conventional glucose measurement apparatus with the rear part thereof detached therefrom;

FIG. 3 is an exploded perspective view showing the construction of a conventional patch for extracting glucose;

FIG. 4 is an exploded perspective view showing the construction of a patch for extracting glucose according to an embodiment of the present invention;

FIG. 5 is a view showing the shape of a first electrode film according to an embodiment of the present invention;

FIGS. 6 and 7 are views showing the construction of the electrode films of a patch for extracting glucose according to another embodiment of the present invention;

FIG. 8 is a sectional view showing a patch for extracting glucose according to an embodiment of the present invention;

FIG. 9 is a view showing a shape in which first and second electrode films are adhered to each other according to an embodiment of the present invention; and

FIG. 10 is a sectional view showing a first electrode film, a second electrode film and a support according to an embodiment of the present invention.

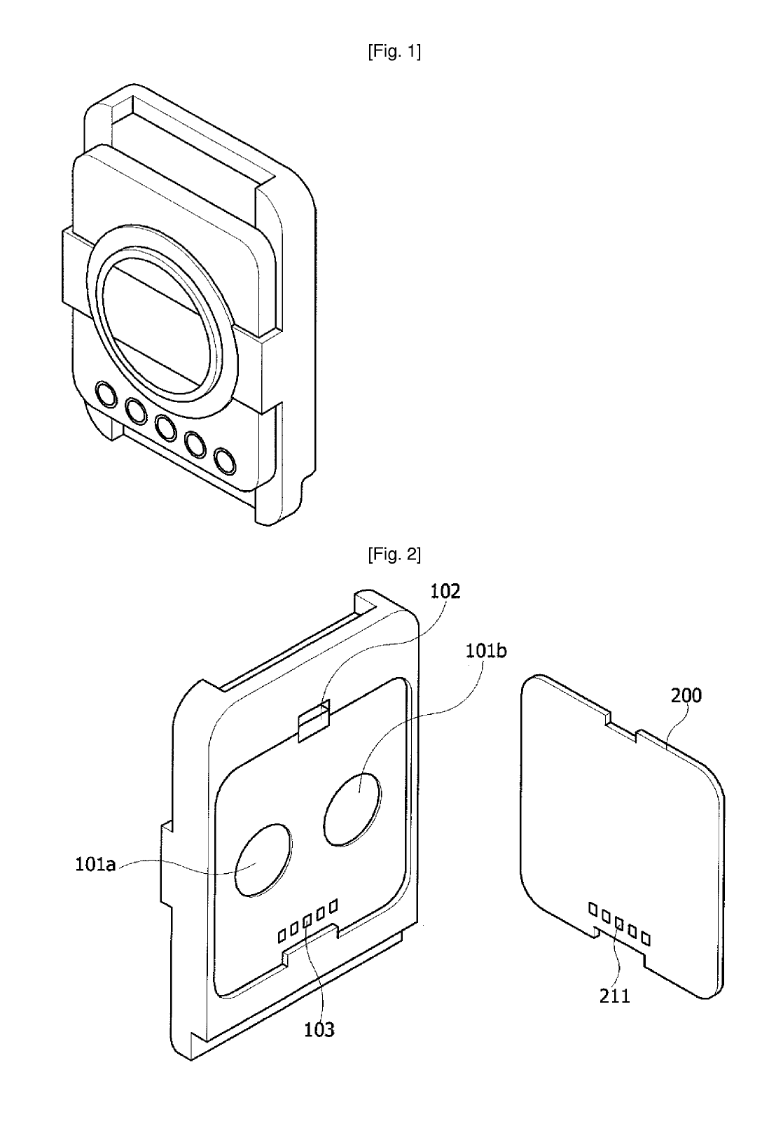

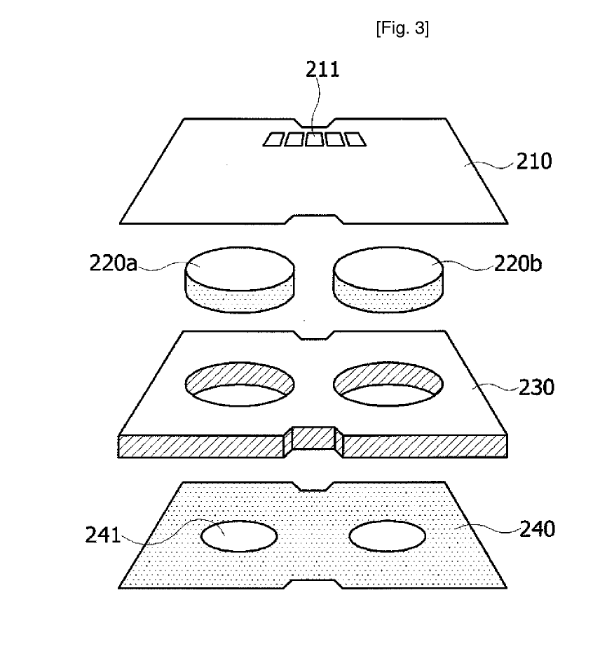

### DESCRIPTION OF REFERENCE CHARACTERS OF IMPORTANT PARTS

101a, 101b: battery receiving part 102: locker
103: main body terminal 200: disposable patch
211: patch terminal 210: print circuit
220a, 220b: gel disc 230: frame
240: film 241: hole
310: frame 320: first electrode film
330a, 330b: hydrophilic gel element 340: nanofiber mesh sheet
350: second electrode film 360: support
312a, 312b: protrusion 321: counter electrode
322: reference electrode 323, 324: hole
325: copper wire 326: terminal
351: first extraction electrode 352: second extraction electrode
353: working electrode 354: copper wire
355: terminal

### BEST MODE FOR CARRYING OUT THE INVENTION

Hereinafter, embodiments of the present invention will be described in detail with reference to the attached drawings. Reference now should be made to the drawings, in which the same reference numerals are used throughout the different drawings to designate the same or similar components. Detailed descriptions may be omitted if it is determined that the detailed descriptions of related well-known functions and construction may make the gist of the present invention unclear.

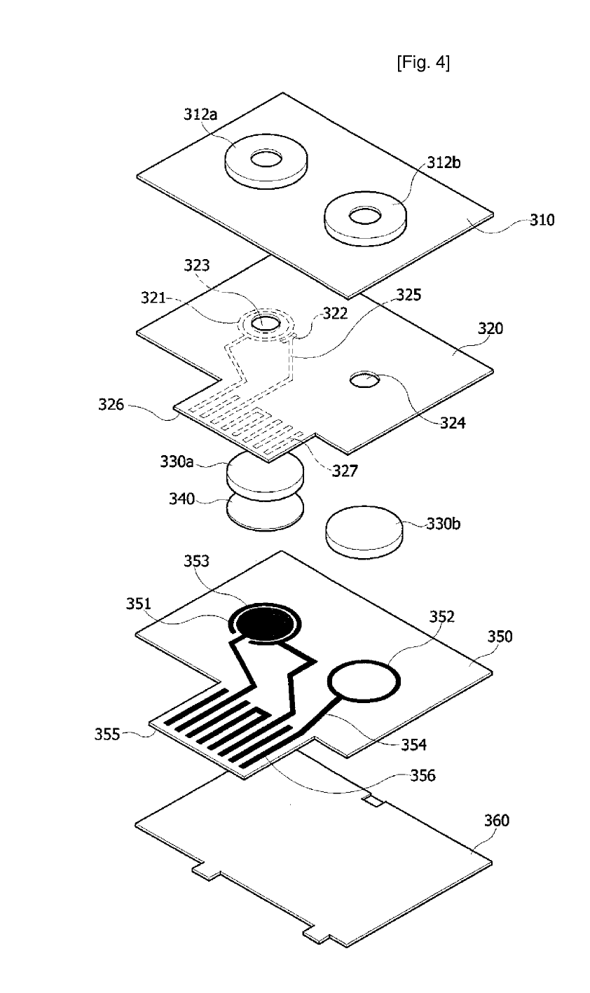

FIG. 4 is an exploded perspective view showing the construction of a patch for extracting glucose according to an embodiment of the present invention. In FIG. 4, the glucose extraction patch includes a frame 310, a first electrode film 320, hydrophilic gel elements 330a and 330b, a nanofiber mesh sheet 340, a second electrode film 350, and a support 360.

The frame 310 includes two protrusions 312a and 312b formed thereon. In the center portions of the protrusions, contact holes coming into contact with the skin are formed, respectively. A polyethylene (PE) material can be used as the material of the frame 310.

The first electrode film 320 is attached to the inner side of the frame 310 in the same shape as that of the frame 310, has holes 323 and 324 formed at locations corresponding to the contact holes, respectively, and includes electrodes 321 and 322 for extracting glucose, a terminal 327 for realizing electrical connection, and copper wires 325 for connecting the electrodes 321 and 322 to the terminal 327. A polyethylene (PE) material can be used as the material of the first electrode film 320.

In an embodiment of the present invention, the first electrode film 320 has the same shape as the frame 310. There may be two methods of manufacturing the shape. A first method is to manufacture the first electrode film 320 in the shape of the frame 310 from the beginning when the first electrode film 320 is manufactured. A second method is to primarily manufacture the first electrode film 320 in a planar shape, and to press the first electrode film 320 on the frame 310 in a press manner in a subsequent manufacturing procedure, thus completing the shape of the first electrode film 320.

Media are respectively accommodated in the protrusions 312a and 312b, and have ionic conductivity for the movement of ions and hydrophilic properties. At least one of the media includes enzymes reacting with glucose to generate ions.

The present invention proposes two embodiments for the shape of the medium including enzymes. A first embodiment is constructed to implement the medium using only a hydrophilic gel element 330a or 330b including enzymes, as disclosed in Korean Patent Registration No. 10-453-483, which is the prior art. In this embodiment, at least one of the two hydrophilic gel elements 330a and 330b must have enzymes.

A second embodiment for the shape of the medium including enzymes is constructed in such a way that a nanofiber mesh sheet 340, having enzymes attached thereto, is adhered to the bottom of a hydrophilic gel element 330a having no enzymes.

In this case, the remaining hydrophilic gel element 330b may have enzymes or not. In this case, a method of attaching enzymes to the nanofiber mesh sheet 340 is described in detail. In order to attach enzymes, a cross-linking agent and an enzyme solution must be stirred together. A method of preparing a cross-linking agent is performed by inserting macromolecules capable of forming chains, such as polyvinyl resin adhesive (PVA), polyacrylonitrile, nylon, cellulose or polyvinyl alcohol, into a phosphate buffer solution (PBS) at a weight concentration of 5 to 35 wt % (preferably, 10 wt %), and by dissolving the macromolecules while stirring the macromolecules into the PBS solution within the temperature range of 24 to 25° C. for 3 to 4 hours. As the PBS solution used herein, a 1 Mol PBS solution having pH 5.1 to 7.4 (preferably, pH 7.3) is used. For example, a 1 Mol PBS solution having pH 7.4 is prepared by mixing a 1 Mol KH₂PO₄ solution with a 1 Mol K₂HPO₄ solution, causing a resulting solution to have pH 7.4, and mixing 0.14 Mol NaCl with the resulting solution having pH 7.4. Enzymes are inserted into the cross-linking agent solution, prepared in this way, to obtain a concentration of 5 to 50 mMol (preferably, 10 mMol). Thereafter, the enzymes are dissolved into the cross-linking agent solution while being stirred into the cross-linking agent solution at a temperature of 20° C. for about 30 minutes.

The second electrode film 350 includes electrodes 351, 352 and 353 formed at locations corresponding to those of the two media to extract glucose, a terminal 356 for realizing electrical connection, and copper wires 354 for connecting the electrodes 351, 352 and 353 to the terminal 356. A polyethylene (PE) material can be used as the material of the second electrode film 350.

The support 360 is attached to the second electrode film 350, and is used for physical connection to a glucose measurement apparatus.

FIG. 8 is a sectional view showing the embodiment of FIG. 4. As shown in FIG. 8, the glucose extraction patch of the present invention has a structure in which the support 360, the second electrode film 350, the nanofiber mesh sheet 340, the hydrophilic gel elements 330a and 330b, the first electrode film 320, and the frame 310 are sequentially adhered to each other.

Next, the construction of respective electrodes of the first electrode film 320 and the second electrode film 350 is described below in detail.

First, the first electrode film 320 is described. The electrodes of the first electrode film 320 includes a reference electrode 322 used as a reference potential when current, generated by the reaction of the media with extracted glucose, is measured, and a counter electrode 320 used to measure current, generated by the reaction of the media with the extracted glucose.

In FIG. 4, the counter electrode 321 is formed in the shape of a ring in which a hole 323 corresponding to a contact hole is formed at the center of the ring, and is placed at a location corresponding to the hydrophilic gel element 330a. Further, the reference electrode 322 is formed in the shape of a bar curved along the outer side of the counter electrode 321.

Next, the electrodes of the second electrode film 350 include first and second extraction electrodes 351 and 352 for extracting glucose from the skin, and a working electrode 353 for applying voltage that is used to measure current generated by the reaction of the media with the extracted glucose.

In FIG. 4 the working electrode 353 is formed in the shape of a circle, and is placed at a location corresponding to the counter electrode 321. The first extraction electrode 351 is formed in the shape of a partially cut ring, and is formed to enclose the outer side of the working electrode 353. The second extraction electrode 352 is formed in the shape of a ring, and is placed at a location corresponding to the hydrophilic gel element 330b.

In the embodiment of FIG. 4, the nanofiber mesh sheet 340 is interposed between the working electrode 353 of the second electrode film 350 and the hydrophilic gel element 330a corresponding to the working electrode 353, and is formed in the shape of a circle which has a diameter greater than that of the first extraction electrode 351, the center of the circle being placed at a location coincident with the center of the working electrode 353.

Meanwhile, the arrangement of the electrodes of the first electrode film 320 and the second electrode film 350 in FIG. 4 is only an embodiment, and can be realized in various shapes.

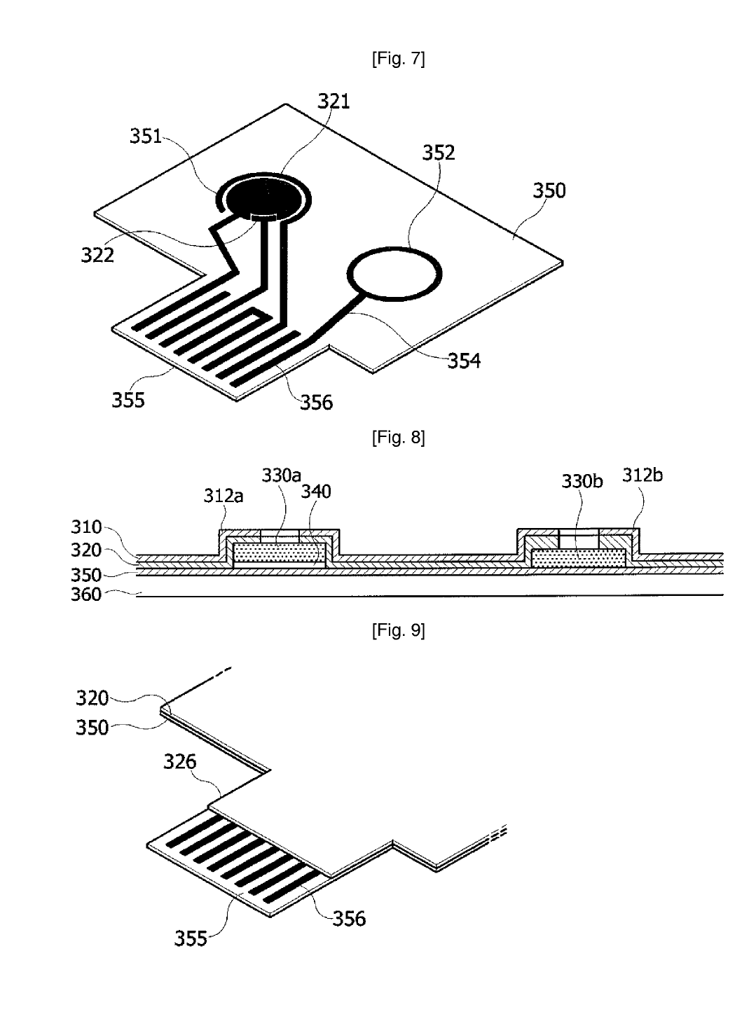

FIGS. 6 and 7 are views showing the construction of the electrodes of the first electrode film 320 and the second electrode film 350 according to another embodiment of the present invention.

FIG. 6 illustrates another embodiment of the electrode of the first electrode film 320. In FIG. 6, the electrode of the first electrode film 320 includes a working electrode 353 for applying voltage that is used to measure current generated by the reaction of the media with the extracted glucose. The working electrode 353 is formed in the shape of a ring, in which a hole corresponding to a contact hole is formed at a center portion, and is placed at a location corresponding to the hydrophilic gel element 330a.

FIG. 7 illustrates another embodiment of the electrodes of the second electrode film 350. In FIG. 7, the electrodes of the second electrode film 350 include a reference electrode 322 used as a reference potential when current generated by the reaction of media with extracted glucose is measured, a counter electrode 321 used to measure current generated by the reaction of the media with the extracted glucose, and first and second extraction electrodes 351 and 352 used to extract glucose from the skin.

In FIG. 7, the counter electrode 321 is formed in the shape of a circle having a partially recessed edge, and is placed at a location corresponding to the working electrode 353 of the first electrode film 320. The reference electrode 322 is placed in the location at which the edge of the counter electrode 321 is partially recessed. The first extraction electrode 351 is formed in the shape of a partially cut ring, and is formed to enclose the outer side of the counter electrode 321. The second extraction electrode 352 is formed in the shape of a ring, and is placed at a location corresponding to the hydrophilic gel element 330b.

In the embodiments of FIGS. 6 and 7, the nanofiber mesh sheet 340 is interposed between the counter electrode 321 of the second electrode film 350 and the hydrophilic gel element 330a corresponding to the counter electrode 321, and is formed in the shape of a circle which has a diameter greater than that of the first extraction electrode 351, the center of the circle being placed at a location coincident with the center of the counter electrode 321.

As shown in the embodiments of FIGS. 4, 6 and 7, electrodes, terminals and copper wires are printed on opposite surfaces of both the first electrode film 320 and the second electrode film 350. A portion of the first electrode film 320, other than the protrusions formed around the holes 323 and 324, is adhered to the second electrode film 350. That is, the portion of the first electrode film 320, other than the protrusions, is adhered to the second electrode film 350 so that the terminal 327 of the first electrode film 320 and the terminal 356 of the second electrode film 350 are electrically connected to each other.

In the embodiment of FIG. 4, a portion of the first electrode film 320, in which the terminal 327 is placed, protrudes from the remaining portion of the first electrode film 320, thus forming a terminal protrusion 326. Further, the portion of the second electrode film 350, in which the terminal 356 is placed, protrudes from the remaining portion of the second electrode film 350, thus forming a terminal protrusion 355. As shown in FIG. 9, it can be seen that the terminal protrusion 355 of the second electrode film 350 protrudes further than the terminal protrusion 326 of the first electrode film 320.

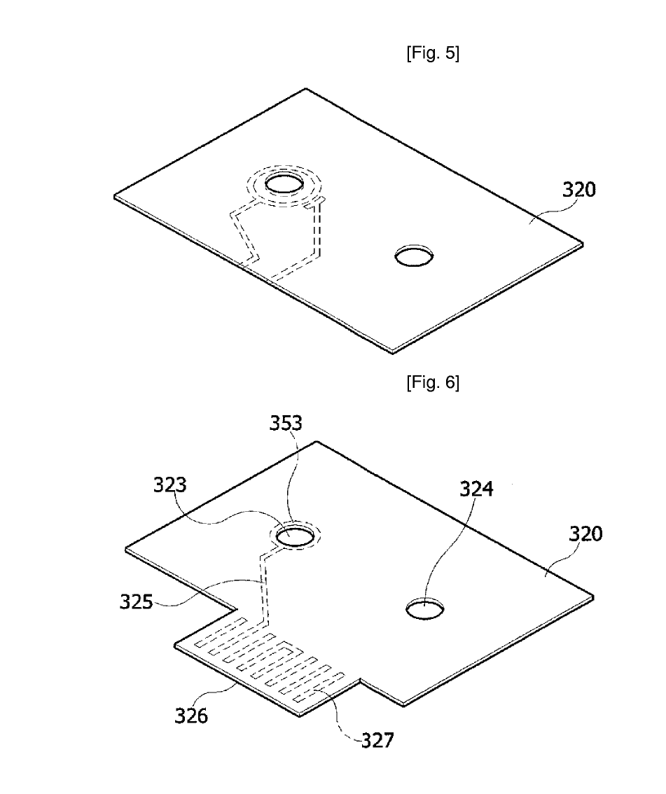

Meanwhile, according to an embodiment, the terminal protrusion 326 of the first electrode film 320 can be removed. In this case, the shape of the first electrode film 320 is shown in FIG. 5.

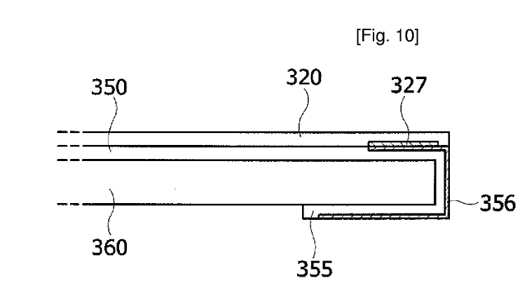

FIG. 10 is a sectional view showing the first electrode film 320, the second electrode film 350 and the support 360 according to an embodiment of the present invention. As shown in FIG. 10, the first electrode film 320 and the second electrode film 350 are adhered to each other, with the terminals 327 and 356 coming into contact with each other. The terminal protrusion 355 of the second electrode film 350 is formed such that it is bent at the edge of the support 360 and is adhered to the bottom surface of the support 360, thus causing the terminal 356 to be exposed from the bottom surface of the support 360. In this way, the patch can be electrically connected to a glucose extraction apparatus through the exposed terminal 356. Meanwhile, for a method of adhering the first electrode film 320 and the second electrode film 350 to each other, a method of applying an adhesive to the surface of the film can be used, or an ultrasonic welding or thermal welding method can be used.

Although the preferred embodiments of the present invention have has been disclosed for illustrative purposes, those skilled in the art will appreciate that various modifications, additions and substitutions are possible, without departing from the scope and spirit of the invention as disclosed in the accompanying claims.

### INDUSTRIAL APPLICABILITY

As described above, according to the present invention, the electrodes of a glucose extraction patch are arranged using two films, so that there are advantages in that conductivity and sensitivity are improved, and the analysis time required for the extraction of glucose can be reduced.

1. A patch for extracting glucose, comprising:
a frame including two protrusions formed thereon, the two protrusions having contact holes that are formed at center portions thereof and come into contact with skin;
a first electrode film attached to an inner side of the frame in a same shape as that of the frame, and provided with holes formed at locations corresponding to the contact holes, the first electrode film including a electrode for extracting glucose, a terminal for realizing electrical connection, and copper wires for connecting the electrodes to the terminal;
two media accommodated in the protrusions, respectively, and adapted to have ionic conductivity for movement of ions and hydrophilic properties, at least one of the two media including enzymes required to generate ions through reaction with glucose;
a second electrode film including electrodes placed at locations corresponding to the two media, respectively, to extract glucose, a terminal for realizing electrical connection, and copper wires for connecting the electrodes to the terminal; and
a support attached to the second electrode film and required for physical connection to a glucose measurement device.

2. The patch according to claim 1, wherein:
the first electrode film comprises a reference electrode used as a reference potential when current generated by reaction of the media with extracted glucose is measured, and a counter electrode used to measure current generated by reaction of the media with extracted glucose, and
the electrodes of the second electrode film comprise first and second extraction electrodes for extracting glucose form skin, and a working electrode for applying voltage that is used to measure current generated by reaction of the media with extracted glucose.

3. The patch according, to claim 2, wherein;
the counter electrode is formed in a shape of a ring, in which a hole corresponding to one of the contact holes is formed at a center portion, and is placed at a location corresponding to one of the media;
the reference electrode is formed in a shape of a bar that is curved along all outer side of the counter electrode,
the working electrode is formed in a shape of a circle, and is placed at a location corresponding to the counter electrode,
the first extraction electrode is formed iii a shape of a partially cut ring, and is formed to enclose an outer side of the working electrode, and
the second extraction electrode is formed in a shape of a ring, and is placed at a location corresponding to a medium, other than the medium corresponding to the counter electrode.

4. The patch according to claim 3, wherein the at least one medium including enzymes comprises a hydrophilic gel element and a nanofiber mesh sheet adhered to a bottom of the hydrophilic gel element and having enzymes attached thereto.

5. The patch according to claim 4, wherein the nanofiber mesh sheet is formed in a shape of a circle having a diameter greater than that of the first extraction electrode, a center of the circle being, placed at a location coincident with a center of the working electrode.

6. The patch according to claim 1, wherein:
the electrode of the first electrode film comprises a working electrode for applying voltage that is used to measure current generated by reaction of the media with extracted glucose, and
the electrodes of the second electrode film comprise a reference electrode used as a reference potential when current generated by reaction of the media with extracted glucose is measured, a counter electrode used to measure current generated by reaction of the media with extracted glucose, and first and second extraction electrodes used to extract glucose from skin.

7. The patch according to claim 6, wherein the working electrode is formed in a shape of a ring, in which a hole corresponding to one of the contact holes is formed at a center portion, and is placed at a location corresponding to one of the media,
the counter electrode is formed in a shape of a circle having a partially recessed edge, and is placed at a location corresponding to the working electrode;
the reference electrode is placed iii a location at which the edge of the counter electrodes is partially recessed,
the first extraction electrode is formed in a shape of a partially cut ring, and is formed to enclose an outer side of the counter electrode, and
the se-coned extraction electrode, is formed in a shape of a ring, and is placed at a location corresponding to a medium, other that the medium corresponding to the working electrode.

8. The patch according to claim 7, wherein the at least one medium including enzymes comprises a hydrophilic gel element and a nanofiber mesh sheet adhered to a bottom of the hydrophilic gel element and having enzymes attached thereto.

9. The patch according to claim 8, wherein the nanofiber mesh sheet is formed in a shape of a circle having a diameter greater than that of the first extraction electrode, a center of the circle being, placed at a location coincident with a center of the working electrode.

10. The patch according to claim 1, wherein the first electrode film and the second electrode film are constructed so that the electrodes, the terminals and the copper wires are printed on opposite surfaces thereof, and a portion of the first electrode film, other than the protrusions, is adhered to the second electrode film.

11. The patch according to claim 10, wherein the portion of the first electrode film, other than the protrusions, is adhered to the second electrode film so that the terminal of the first electrode film is electrically connected to the terminal of the second electrode film.

12. The patch according to claim 1 wherein a portion of the first electrode film, in which the terminal is placed, protrudes from a remaining portion of the first electrode film, thus forming a terminal protrusion, and a portion of the second electrode film, in which the terminal is placed, protrudes form a remaining portion of the second electrode film, thus forming a terminal protrusion.

13. The patch according to claim 12, wherein the terminal protrusion of the second electrode film protrudes further than the terminal protrusion ion of the first electrode film.

14. The patch according to claim 13, wherein the portion of the second electrode film, in which the terminal is placed, is bent at an edge portion of the support and is adhered to a bottom surface of the support, thus causing the terminal to be exposed from the back surface of the support.
